(19) United States
(12) Patent Application Publication
(10) Pub. No.: US 2011/0229882 A1
(43) Pub. Date: Sep. 22, 2011

(54) DIAGNOSING AND MONITORING RESPONSE TO TREATMENT OF SOLID ORGAN TISSUE DISEASE

(75) Inventors: Martin Heath Bluth, Southfield, MI (US); Wellman Cheung, Briarwood, NY (US)

(73) Assignees: Martin Heath Bluth, Southfield, MI (US); Wellman Cheung, Briarwood, NY (US)

(21) Appl. No.: 13/130,351

(22) PCT Filed: Nov. 20, 2009
(86) PCT No.: PCT/US09/65394

§ 371 Date: May 20, 2011

Related U.S. Application Data
(60) Provisional application No. 61/116,777, filed on Nov. 21, 2008.
(60) Provisional application No. 61/116,791, filed on Nov. 21, 2008.
(60) Provisional application No. 61/116,794, filed on Nov. 21, 2008.
(60) Provisional application No. 61/116,796, filed on Nov. 21, 2008.

(51) Int. Cl. C12Q 1/68

(52) U.S. Cl. 435/6.1

(57) ABSTRACT

Techniques for diagnosing and monitoring response to treatment of a solid organ tissue disease in a patient are provided. For example, a technique for diagnosing a solid organ tissue disease in a patient includes obtaining at least one of one or more blood-derived nucleated acellular components and one or more nucleated cellular components from the patient to provide a reporter function in the patient. Also, a technique for monitoring response to treatment of a solid organ tissue disease in a patient includes obtaining at least one of one or more blood-derived nucleated acellular components and one or more nucleated cellular components from the patient to provide a reporter function in the patient.

### CROSS-REFERENCE TO RELATED APPLICATION(S)

This application claims the benefit of U.S. provisional application Ser. No. 61/116,777, filed on Nov. 21, 2008, U.S. provisional application Ser. No. 61/116,791, filed on Nov. 21, 2008, U.S. provisional application Ser. No. 61/116,794, filed on Nov. 21, 2008, and U.S. provisional application Ser. No. 61/116,796, filed on Nov. 21, 2008, the disclosures of which are incorporated by reference herein in their entireties for all purposes.

### FIELD OF THE INVENTION

The present invention relates generally to immunology, and more particularly relates to diagnosing and monitoring response to treatment of solid organ disease.

### BACKGROUND OF THE INVENTION

Malignancy, also referred to as cancer, affecting solid organs continues to prove fatal and affects both men and women. For example, pancreatic cancer often presents in late disease stage as it is largely undetected until presentation of symptoms. In addition, pancreatic cancer can remain undiagnosed or untreated, due, in large part, to the lack of early detection and invasive existing diagnostic approaches. Furthermore, pancreatic cancer can be confused with other diseases presenting with obstructive jaundice, making the correct diagnosis in these patients very difficult.

Pancreatic cancer continues to present diagnostic challenges, and confirmation of disease often requires invasive procedures. For example, pancreatic cancer is generally diagnosed by history and/or confirmed by invasive technology such as pancreatic tissue biopsy. These tests are hampered by the need to effectively obtain the cancerous tissue, a procedure involving exploratory abdominal surgery and/or ultrasound/imaging-guided biopsy. In each case, procurement of appropriate tissue for correct histological diagnosis is not assured and can lead to misdiagnosis. Additionally, such conventional procedures are invasive and carry additional morbidity and mortality risk, and are therefore undesirable.

### SUMMARY OF THE INVENTION

The present invention, in illustrative embodiments thereof, provides techniques for diagnosing solid organ tissue disease and/or monitoring response to treatment of such diseases (for example, pancreatic cancer).

In accordance with one aspect of the invention, techniques for diagnosing a solid organ malignancy in a patient includes obtaining blood-derived nucleic acid containing nucleated acellular components such as serum and/or plasma and/or cellular components such as leukocytes including polymorphonuclear cells (PMN), peripheral blood mononuclear cells (PBMC), basophils, eosinophils, reticulocytes and/or platelets from the patient to provide a reporter function in the patient.

Additionally, in accordance with another aspect of the invention, techniques for monitoring response to treatment of solid organ malignancy in a patient includes obtaining blood-derived nucleic acid containing nucleated acellular components such as serum and/or plasma and/or cellular components such as leukocytes including polymorphonuclear cells (PMN), peripheral blood mononuclear cells (PBMC), basophils, eosinophils, reticulocytes and/or platelets from the patient to provide a reporter function in the patient. One or more embodiments of the invention can also be used for related and comparative non-malignant diseases of solid tissue such as cystic diseases which can present as malignancy.

These and other features, objects and advantages of the present invention will become apparent from the following detailed description of illustrative embodiments thereof, which is to be read in connection with the accompanying drawings.

### BRIEF DESCRIPTION OF THE DRAWINGS

FIG. 1 is a flow diagram illustrating an exemplary technique for diagnosing a solid organ tissue disease in a patient, according to an embodiment of the present invention;

FIG. 2 is a flow diagram illustrating an exemplary technique for monitoring response to treatment of solid organ tissue disease in a patient, according to an embodiment of the present invention; and

FIG. 3 is a flow diagram illustrating an exemplary technique for prognosticating a solid organ tissue disease in a patient, according to an embodiment of the present invention.

### DETAILED DESCRIPTION OF PREFERRED EMBODIMENTS

Initially, the complete disclosures of U.S. provisional patent application Ser. No. 61/116,777 to Bluth et al. entitled “Monitoring Response to Treatment of Urological Disease,” filed on Nov. 21, 2008, U.S. provisional patent application Ser. No. 61/116,791 to Bluth et al. entitled “Diagnosing and Monitoring Response to Treatment of Pancreatic Disease,” filed on Nov. 21, 2008, U.S. provisional patent application Ser. No. 61/116,794 to Bluth et al. entitled “Diagnosing and Monitoring Response to Treatment of Benign Prostatic Hypertrophy,” filed on Nov. 21, 2008, and U.S. provisional patent application Ser. No. 61/116,796 to Bluth et al. entitled “Diagnosing and Monitoring Response to Treatment of Erectile Dysfunction,” filed on Nov. 21, 2008, are expressly incorporated herein by reference in their entireties for all purposes.

It should be understood that one or more embodiments of the invention can be applied to all manner of solid organ malignancies including, but not limited to, pancreas, prostate, liver, gut, lung, breast and skin, among others. The term applying to “solid organ diseases” as used herein is intended to contrast with respect to malignancies of, for example, leukemia or lymphoma type. The techniques described herein can also be used for related and comparative non-malignant diseases of solid tissue, such as, for example, cystic diseases, which can present as malignancy. Therefore, such a platform can help distinguish malignancy from non-malignancy with regard to biomarker discovery. By way of example only and without loss of generality, pancreatic cancer, as noted above, presents diagnostic difficulties and confirmation of disease often requires invasive approaches. As such, a blood-based assay with appropriate diagnostic sensitivity and specificity would complement or obviate these invasive testing procedures and would therefore be advantageous.

As described herein, nucleic acid containing nucleated acellular components such as serum and/or plasma and/or nucleated cells derived from whole blood, including leukocytes such as polymorphonuclear (PMN) cells, peripheral blood mononuclear cells (PBMC) eosinophils, basophils, reticulocytes and platelets can provide a reporter function in solid organ retroperitoneal disease (for example, pancreas disease). PMN cells are also referred to as granulocytes and PBMC include mononucleated cells consisting of B cells, T cells, monocytes, and natural killer cells, among others. As such, all of these nucleated acellular and cellular nucleic acid containing components contain ribonucleic acid (RNA) which provides the translational message for proteins. This RNA can be interrogated for biomarker discovery. One or more embodiments of the invention use nucleic acid (RNA) containing nucleated acellular components (also referred to as nucleated components) such as serum and/or plasma and/or nucleated cells derived from whole blood which include leukocytes such as PMN cells, PBMC eosinophils, basophils, reticulocytes and/or platelets as a marker for patients with solid organ malignancy and use nucleic acid (RNA) containing nucleated acellular components such as serum and/or plasma and/or nucleated cells derived from whole blood which include leukocytes such as PMN cells, PBMC eosinophils, basophils, reticulocytes and platelets as a marker for patients with confirmed malignancy.

It is further understood that non-malignant diseases that can be confused with malignant disease at presentation or after further workup, such as cystic disease, are also to be considered under the scope of one or more embodiments of the invention as a means to differentiate malignant from non-malignant (cancer versus precancerous or benign) with respect to biomarker discovery.

The teem “patient” as used herein is intended to refer broadly to mammalian subjects, and more preferably refers to humans receiving medical attention (e.g., diagnosis, monitoring, etc.), care or treatment.

With respect to one or more embodiments of the invention, proof of concept is derived from similar studies utilizing said techniques applied to pancreatitis and overactive bladder (OAB) as a model for solid organ disease. Similarly, solid organ malignancy (for example, pancreatic cancer) represents solid organ diseases where the afflicted organ (e.g., pancreas) is intracavitary, composed of solid tissue and is not easily accessible. The following exemplary data derived, by one skilled in the art, from pancreatitis can easily be derived for malignancies or relative non-malignancy affecting other solid organs, given the teachings herein.

By way of example, one or more embodiments of the invention find the same gene product (for example, PDGF-C) in PMN cells (cellular) and serum/plasma (acellular) components. By way of a nexus, PBMC are comprised of many types of immune cells such as B cells, T cells, monocytes etc. As such, PMN cells, platelets and reticulocytes represent other types of immune cells or related cells which can provide additional reporter function. Further, serum and plasma can contain the nucleic acid such as RNA normally contained within the nucleated cells (PBMC, PMN cells, etc.) and, as such, can be interrogated directly to provide a reporter function of immune or immune-related cells without the need for obtaining the immune cells directly.

Acute pancreatitis was induced using retrograde infusion of 4% sodium taurocholate (NaT) into the pancreatic ducts of rats. Briefly, under pentobarbital anesthesia (50 milli-gram/kilo-gram (mg/kg) given intraperitoneally), a midline incision was performed. The bile duct was then ligated to prevent the flow of bile and 4% NaT in sterile saline was infused into the pancreatic duct at a rate of 1 ml/kg over 10 minutes.

Sepsis, used as a comparative inflammatory disease, was induced by cecal ligation and puncture (CLP). Briefly, under pentobarbital anesthesia, a laparotomy was performed (the size of the incision was 2.5 centimeters (cm)), and the cecum was ligated just below the ileocecal valve with a 3-0 silk ligature and the antimesentric cecal surface was punctured once with a 16-gauge needle proximal to the ligature. The cecum was then returned to the peritoneal cavity and fecal content in the ligated segment was allowed to extrude through the puncture to the peritoneum. The peritoneum and abdominal muscles were closed with silk sutures. After CLP, rats were returned to cages and allowed ad libitum access to food and water.

For both experimental models of acute pancreatitis and sepsis, control rats were anesthetized and sham operated with a laparotomy. The pancreas or cecum was manipulated but neither pancreatitis induction nor CLP procedure was performed.

Twenty-four hours after pancreatitis, septic shock induction, sham operation with saline infusion or in fasted untouched, normal control animals (n=3 each group, n=12 total), approximately 8-10 milli-liters (mL) of whole blood were collected via inferior vena cava (IVC) from each rat under pentobarbital anesthesia. In one or more embodiments of the invention, for example, between one and six mL of whole blood can be collected. PBMC were isolated from rat whole blood by centrifugation through Ficoll-Paque.

Briefly, total RNA was extracted from PBMC using TRIzol and eluted using an RNeasy spin column. Ten micrograms of total RNA was converted into double-stranded cDNA by reverse transcription. The double-strand cDNA product was extracted with phenol/chloroform/isoamyl alcohol using phase lock gels. Double-strand cDNA was, in vitro, transcribed into cRNA and nucleotides were biotinylated. The in vitro transcription product was further purified using RNeasy mini columns and fragmented. The fragmented in vitro transcription product was hybridized onto the rat genome U34A DNA GeneChip Array which contains approximately 7,000 full-length sequences and 1,000 expressed sequence tag (EST) clusters. The sequences were selected from the UniGene database. All 12 PBMC samples were subjected to RNA extraction and transcript profiling.

An absolute expression analysis was performed using Affymetrix MAS 5.0, and the data from genes were imported into GeneSpring software version 5.1 for further analyses. Differentially expressed genes were selected. Differential expression was defined as a change of at least two fold versus respective controls. Non-parametric test was used, assuming non-equal means, specifically the Welch t-test and Welch ANOVA. Significance level was set at >2-fold change between groups, P<0.05.

Rats treated with NaT showed pancreatic edema and necrosis, a hallmark of pancreatitis. Also, the gene transcription profiles of PBMCs in normal, untouched control animals were compared to those with acute pancreatitis, to identify those genes induced in pancreatitis. From the 8,799 rat gene analyzed on the chip, using supervised cluster analysis, 947 genes significantly changed by 2-fold in pancreatitis.

These 947 genes were then subjected to comparison between animals with acute pancreatitis and saline controls, and 170 genes were identified that changed expression. Similarly, 201 unique genes were identified between rats with cecal ligation and puncture (abdominal sepsis) and saline controls, and 409 differentially expressed genes were identified between animals with acute pancreatitis and intra-abdominal sepsis.

Further, of the 170 genes which changed between pancreatitis and saline controls, 15 overlapped when compared to septic/pancreatitic animals, and another 18 overlapped with sepsis/normal animals. In total, 140 genes were unique to PBMCs in animals with pancreatitis. Among the 140 genes whose expression uniquely changed in pancreatitis alone, 57 were upregulated, while 69 were downregulated, and 25% corresponded to ESTs.

As described herein, the genetic profile of pancreas obtained from animals induced with pancreatitis were compared with pancreata from normal, non-operated controls. This identified 947 genes induced during pancreatitis. In order to determine which of these genes were uniquely expressed during pancreatitis, the PBMC RNA obtained from rats induced with pancreatitis were compared with PMBC RNA obtained from rats which underwent saline infusion alone, and with rats with intra-abdominal sepsis which served as comparative inflammatory state.

One hundred forty unique genes were identified which were induced or inhibited in PBMCs during NaT-induced (necrotizing) pancreatic disease. Some of the highly induced genes are of cytokines previously implicated in pancreatitis, such as receptors for platelet-derived factor, transforming growth factor-beta, and a variety of G-protein related signal transduction genes. For example, phospholipase D gene 1 is involved in the intracellular modulation of cellular mitogenesis and even pancreatic organ regeneration. The prostaglandin E2 receptor is induced, which is of note because inhibition of PGE2 by cyclo-oxygenase inhibitors improves survival.

Also, genes associated with cell death, such as caspase 1 and BH3 interacting domain 3, and cell membrane integrity were uniquely downregulated in PBMCs of rats with acute pancreatitis. Caspase 1, associated with cellular apoptosis, its activation within the pancreas, is associated with severe necrosis, but its inhibition may be protective in sepsis. BH3 domain proteins are also involved in mitochondrial-mediated cell death via BCL-2 mediate apoptosis. Additionally, some genes associated with the disease of pancreatitis, namely glucocorticoid receptor, cholecystokinin receptor and lipase are significantly inhibited, each by 7-fold.

As illustrated herein, in acute pancreatitis, PBMCs express genes which are related to pancreatic illness and not intra-abdominal sepsis. As such, the ability of easily accessible acellular blood components such as serum and/or plasma and/or and nucleated cellular components such as leukocytes, reticulocytes and platelets to provide a “reporter function” for solid organ disease will provide usefulness in pancreatic diseases. Furthermore, nucleic acid containing nucleated acellular blood-derived components such as serum and/or plasma also contain RNA which can be interrogated for biomarker discovery in solid organ malignancy. Furthermore, one or more embodiments of the invention include identifying genes involved in the pathogenesis of disease. Also, mapping the expression pattern of these genes in the clinical arena will likely help differentiate the patients who are suffering from mild, moderate and severe solid organ disease, including necrosis and systemic complications.

With the ability to correlate solid tissue and blood-based gene changes, blood-derived nucleated acellular components such as serum and/or plasma and/or nucleated cellular component gene screens can mirror diseased solid tissue gene changes. Also, in one or more embodiments of the invention, the use of blood-derived nucleated acellular components such as serum and/or plasma, and/or nucleated cellular component-derived microarray gene changes can also be censored to highlight gender-specific responses. Such techniques can further elucidate sex-specific mechanisms and provide novel therapeutic possibilities.

As described herein, one or more embodiments of the present invention include non-invasive, safe techniques for providing biomarkers (that is, substances used as indicators of one or more biologic states) for disease when compared with biopsy of solid organ. A biomarker can include, for example, a substance whose detection indicates a particular disease state. More specifically, a biomarker can indicate a change in expression or state of a protein that correlates with the risk or progression of a disease, or with the susceptibility of the disease to a given treatment.

One or more embodiments of the invention facilitate the identification of genes (for example, previously unknown and/or unanticipated genes) involved in solid organ disease, as well as benefiting drug design and development. Additionally, one or more embodiments of the invention identify differentially expressed genes in response to disease severity and/or drug treatment over time, which can provide mechanisms of drug action and further understanding of disease pathophysiology.

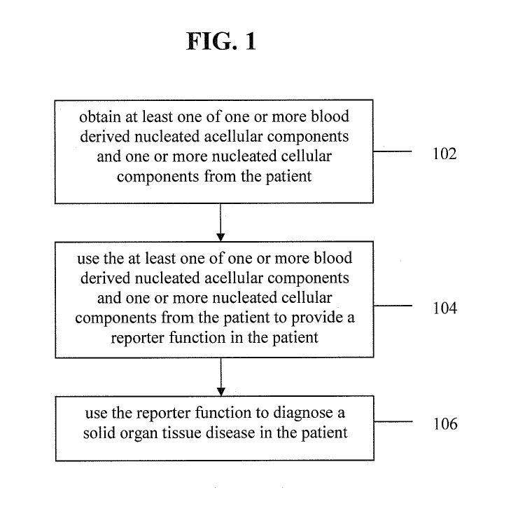

FIG. 1 is a flow diagram illustrating an exemplary methodology for diagnosing a solid organ tissue disease in a patient, according to an embodiment of the present invention. As apparent from the figure, step 102 includes obtaining at least one of one or more blood-derived nucleated acellular components (such as serum and/or plasma) and one or more nucleated cellular components from the patient. Step 104 includes using the at least one of one or more blood-derived nucleated acellular components and one or more nucleated cellular components obtained from the patient to provide a reporter function in the patient. Step 106 includes using the reporter function to diagnose a solid organ tissue disease in the patient.

In one or more embodiments of the invention, the patient includes a patient devoid of an immunomodulatory condition and immunomodulatory therapy. The diseases detailed herein are not generally thought to be of an immunological nature versus inflammatory disease. As such, the selected patients are preferably those which do not have immunomodulatory conditions or immunomodulatory therapy. This allows for biomarker discovery by interrogating the immune or immune-related system in a relatively pure disease state without confounding immunomodulatory activity.

One or more embodiments of the invention include diagnosing a solid tissue malignancy or relative non-malignancy (for example, pancreatic cancer versus non-cancer) in a patient. Obtaining blood-derived nucleated acellular components such as serum and/or plasma and/or nucleated cellular components from a patient can include obtaining whole blood from a patient (for example, in an amount in a range of about four to six milliliters (ml)), and isolating blood-derived nucleated acellular components such as serum and/or plasma and/or nucleated cellular components (for example, polymorphonuclear (PMN) cells, PBMC, eosinophils, basophils, reticulocytes or platelets) from the whole blood.

Also, providing a reporter function in the patient can include, but is not limited to, processing the blood-derived nucleated acellular components such as serum and/or plasma and/or nucleated cellular components to isolate ribonucleic acid (RNA) for analysis. Such analysis can include, for example, comparing gene changes in the patient versus gene changes in a control sample to provide a screening modality to ascertain one or more genes involved in a pathogenesis of the malignancy.

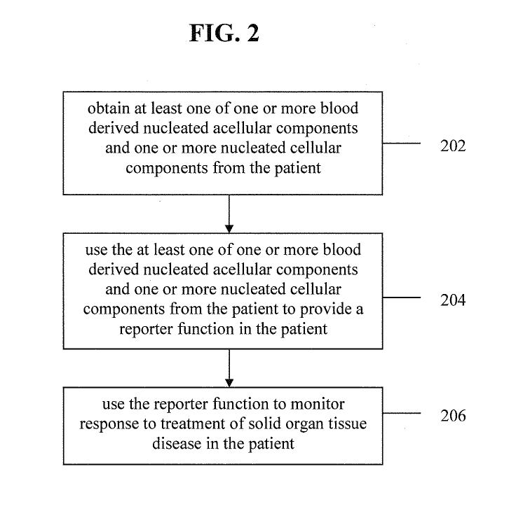

FIG. 2 is a flow diagram illustrating an exemplary methodology for monitoring disease severity and/or response to treatment of solid organ tissue disease in a patient, according to an embodiment of the present invention. Step 202 includes obtaining at least one of one or more blood-derived nucleated acellular components and one or more nucleated cellular components from the patient. Step 204 includes using the at least one of one or more blood-derived nucleated acellular components and one or more nucleated cellular components obtained from the patient to provide a reporter function in the patient. Step 206 includes using the reporter function to monitor response to treatment of solid organ tissue disease in the patient. In one or more embodiments of the invention, the patient includes a patient devoid of an immunomodulatory condition and immunomodulatory therapy.

One or more embodiments of the invention include monitoring response to treatment of solid organ malignancy or related non-malignancy (for example, pancreatic cancer versus non-cancer) in a patient. Obtaining blood-derived nucleated acellular components such as serum and/or plasma and/or nucleated cellular components from a patient can include obtaining whole blood from a patient (for example, in an amount in a range of about four to six milliliters), and isolating blood-derived nucleated acellular components such as serum and/or plasma and/or nucleated cellular components from the whole blood.

Providing a reporter function in the patient can include, for example, processing the blood-derived nucleated acellular components such as serum and/or plasma and/or nucleated cellular components to isolate RNA for analysis, wherein the analysis includes comparing gene changes in the patient versus gene changes in a control sample to provide a screening modality to ascertain one or more genes involved in a pathogenesis of the pancreatic disease. Providing a reporter function can also include ascertaining one or more gene changes with regard to disease severity and/or pre- and post-treatment of malignancy or related non-malignancy in the patient. Further, ascertaining one or more gene changes pre- and post-treatment of malignancy or related non-malignancy in the patient may include elucidating a therapeutic drug mechanism of action.

As noted above, comparing gene changes in a patient versus gene changes in a control samples provides a screening modality to mine for unique genes potentially involved in the pathogenesis of malignancy or related non-malignancy, which can be subsequently confirmed using conventional methods such as, for example, western blot, enzyme linked immunosorbant assay, polymerase chain reaction, etc. The techniques described herein can also determine any gene changes that occur with regard to disease severity and/or pre- and post-therapy, thereby providing a putative mechanism of action for the therapeutic treatment of the one or more malignant or related non-malignancy diseases.

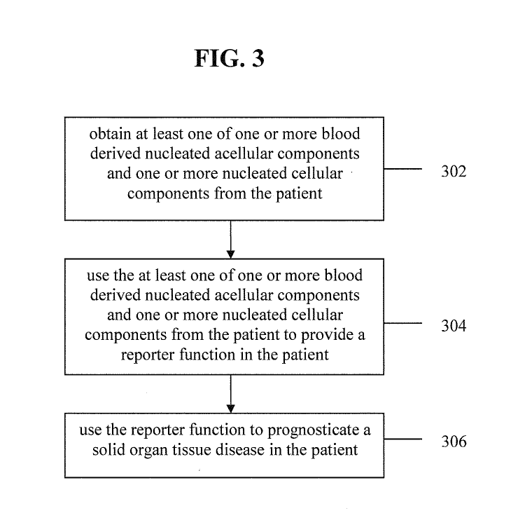

FIG. 3 is a flow diagram illustrating techniques for prognosticating a solid organ tissue disease in a patient, according to an embodiment of the present invention. Step 302 includes obtaining at least one of one or more blood-derived nucleated acellular components and one or more nucleated cellular components from the patient. Step 304 includes using the at least one of one or more blood-derived nucleated acellular components and one or more nucleated cellular components from the patient to provide a reporter function in the patient. Step 306 includes using the reporter function to prognosticate a solid organ tissue disease in the patient.

In one or more embodiments of the invention, the patient includes a patient devoid of an immunomodulatory condition and immunomodulatory therapy. The blood-derived nucleated acellular components can include, for example, serum and/or plasma, and the blood-derived nucleated cellular components can include PMN cells, PBMC, platelets, eosinophils, basophils and/or reticulocytes.

Obtaining blood-derived nucleated acellular components and/or nucleated cellular components from the patient can include obtaining whole blood from the patient, and isolating one or more blood-derived nucleated acellular components and/or one or more nucleated cellular components from the whole blood. Also, providing a reporter function in the patient can include processing the one or more blood-derived nucleated acellular components and/or one or more nucleated cellular components to isolate ribonucleic acid (RNA) for analysis, wherein the analysis includes comparing one or more gene changes in the patient versus one or more gene changes in known disease state samples, including diagnosis, prognosis (disease severity) and pre- and post therapy to provide a screening modality to ascertain severity of the solid organ tissue disease.

Although illustrative embodiments of the present invention have been described herein, it is to be understood that the invention is not limited to those precise embodiments, and that various other changes and modifications may be made by one skilled in the art without departing from the scope or spirit of the invention.

1. A method for diagnosing a solid organ tissue disease in a patient, the method comprising:
obtaining at least one of one or more blood-derived nucleated acellular components and one or more nucleated cellular components from the patient;
using the at least one of one or more blood-derived nucleated acellular components and one or more nucleated cellular components obtained from the patient for providing a reporter function in the patient; and
using the reporter function to diagnose a solid organ tissue disease in the patient.

2. The method of claim 1, wherein the patient comprises a patient devoid of an immunomodulatory condition and immunomodulatory therapy.

3. (canceled)

4. The method of claim 1, wherein the solid organ tissue disease comprises at least one of a malignancy in a solid organ tissue and a related non-malignancy in a solid organ tissue.

5. The method of claim 4, wherein the malignancy comprises cancer of a solid organ tissue.

6. The method of claim 5, wherein the cancer comprises pancreatic cancer.

7. The method of claim 1, wherein the one or more blood-derived nucleated cellular components comprise at least one of polymorphonuclear (PMN) cells, peripheral blood mononuclear cells (PBMC), platelets, eosinophils, basophils and reticulocytes.

8. (canceled)

9. (canceled)

10. The method of claim 1, wherein providing a reporter function in the patient comprises processing the at least one of one or more blood-derived nucleated acellular components and one or more nucleated cellular components to isolate ribonucleic acid (RNA) for analysis, wherein the analysis comprises comparing one or more gene changes in the patient versus one or more gene changes in a control sample to provide a screening modality to ascertain one or more genes involved in a pathogenesis of the solid organ tissue disease.

11. A method for monitoring response to treatment of solid organ tissue disease in a patient, the method comprising:
obtaining at least one of one or more blood-derived nucleated acellular components and one or more nucleated cellular components from the patient;
using the at least one of one or more blood-derived nucleated acellular components and one or more nucleated cellular components obtained from the patient for providing a reporter function in the patient; and
using the reporter function to monitor response to treatment of solid organ tissue disease in the patient.

12. The method of claim 11, wherein the patient comprises a patient devoid of an immunomodulatory condition and immunomodulatory therapy.

13. (canceled)

14. The method of claim 11, wherein the solid organ tissue disease comprises at least one of a malignancy in a solid organ tissue and a related non-malignancy in a solid organ tissue.

15. The method of claim 14, wherein the malignancy comprises cancer of a solid organ tissue.

16. The method of claim 15, wherein the cancer comprises pancreatic cancer.

17. The method of claim 11, wherein the one or more blood-derived nucleated cellular components comprise at least one of polymorphonuclear (PMN) cells, peripheral blood mononuclear cells (PBMC), platelets, eosinophils, basophils and reticulocytes.

18. (canceled)

19. (canceled)

20. The method of claim 11, wherein providing a reporter function in the patient comprises processing the at least one of one or more blood-derived nucleated acellular components and one or more nucleated cellular components to isolate ribonucleic acid (RNA) for analysis, wherein the analysis comprises comparing one or more gene changes in the patient versus one or more gene changes in a control sample to provide a screening modality to ascertain one or more genes involved in a pathogenesis of the solid organ tissue disease.

21. The method of claim 11, wherein providing a reporter function in the patient comprises ascertaining one or more gene changes pre- and post-treatment of the solid organ tissue disease in the patient.

22. The method of claim 21, wherein ascertaining one or more gene changes pre- and post-treatment of the solid organ tissue disease in the patient further comprises elucidating a therapeutic mechanism of action.

23. A method for prognosticating a solid organ tissue disease in a patient, the method comprising:
obtaining at least one of one or more blood-derived nucleated acellular components and one or more nucleated cellular components from the patient;
using the at least one of one or more blood-derived nucleated acellular components and one or more nucleated cellular components obtained from the patient for providing a reporter function in the patient; and
using the reporter function to prognosticate a solid organ tissue disease in the patient.

24. The method of claim 23, wherein the patient comprises a patient devoid of an immunomodulatory condition and immunomodulatory therapy.

25. The method of claim 23, wherein the one or more blood-derived nucleated acellular components comprise at least one of serum and plasma, and the one or more blood-derived nucleated cellular components comprise at least one of polymorphonuclear (PMN) cells, peripheral blood mononuclear cells (PBMC), platelets, eosinophils, basophils and reticulocytes.

26. (canceled)

27. The method of claim 23, wherein providing a reporter function in the patient comprises processing the at least one of one or more blood-derived nucleated acellular components and one or more nucleated cellular components to isolate ribonucleic acid (RNA) for analysis, wherein the analysis comprises comparing one or more gene changes in the patient versus one or more gene changes in known disease state samples to provide a screening modality to ascertain severity of the solid organ tissue disease.
